(19) United States
(12) Patent Application Publication
(10) Pub. No.: US 2013/0345726 A1
(43) Pub. Date: Dec. 26, 2013

(54) SUTURE PULLER

(71) Applicants: Alfred Rodrigue Berube, JR., North Attleboro, MA (US); Gary Robert McCarthy, East Bridgewater, MA (US); Benjamin K. Graf, Madison, WI (US)

(72) Inventors: Alfred Rodrigue Berube, JR., North Attleboro, MA (US); Gary Robert McCarthy, East Bridgewater, MA (US); Benjamin K. Graf, Madison, WI (US)

(73) Assignee: Smith & Nephew, Inc., Memphis, TN (US)

(21) Appl. No.: 13/529,138

(22) Filed: Jun. 21, 2012

(51) Int. Cl. A61B 17/04

(52) U.S. Cl. 606/148

(57) ABSTRACT

A suture puller includes a tubular body member with a first end and a second end. First and second conical end members are coupled to the first and second ends of the tubular body member, respectively. The first conical end member and the tubular body member define a slot, configured to receive a suture, that extends through the first conical end member and a portion of the tubular body member along a central axis of the suture puller.

### TECHNICAL FIELD

This disclosure relates to a suture puller.

### BACKGROUND

Some surgical procedures involve passing sutures through passages in bone. For instance, an anterior cruciate ligament (ACL) that has ruptured and is non-repairable can be replaced by a tissue graft. The replacement tissue graft can be placed by pulling a tissue graft into a bone tunnel via passing sutures.

### SUMMARY

In one aspect, a suture puller includes a tubular body member with a first end and a second end. A first conical end member is coupled to the first end of the tubular body member and a second conical end member is coupled to the second end of the tubular body member. The first conical end member and the tubular body member define a slot that extends through the first conical end member and a portion of the tubular body member along a central axis of the suture puller. The slot is configured to receive a suture.

Implementations may include one or more of the following features. For example, the tubular body member may define a locking slot on a surface of the tubular body member. The locking slot may be configured to receive and hold part of the suture. A majority of the surface of the tubular body member may be textured. The slot may be lined with a material configured to prevent slipping of the suture. The slot may be configured to be compressed such that the compression stops motion of the suture in the slot. The tubular body member may be a cylindrical body member that does not have edges. The suture puller may less than approximately eight inches long from an end of the first conical end member to an end of the second conical end member. The suture puller may be less than approximately 6 inches long from the end of the first conical end member to the end of the second conical end member. The suture puller may be approximately 4.5 inches long from the end of the first conical end member to the end of the second conical end member.

In a general aspect, a method includes forming a bone tunnel, coupling a tissue graft to a graft attachment device, coupling a first end of a suture to the graft attachment device, passing a second end of the suture through the bone tunnel, coupling the second end of the suture to a suture puller, and manipulating the suture puller to draw the suture, graft attachment device, and tissue graft through the bone tunnel. The suture puller including a tubular body member having a first end and a second end, a first conical end member coupled to the first end of the tubular body member, and a second conical end member coupled to the second end of the tubular body member.

Implementations may include one or more of the following features. For example, the first conical end member and the tubular body member may define a slot that extends through the first conical end member and a portion of the tubular body member along a central axis of the suture puller. Coupling the second end of the suture to the suture puller may include placing the second end of the suture into the slot. Coupling the second end of the suture to the suture puller may include wrapping a portion of the suture around the tubular body member after placing the second end of the suture in the slot. The method may include compressing the slot such that the compression stops motion of the suture in the slot. The slot may be lined with a material configured to prevent slipping of the suture. The suture puller may include a locking slot on a surface of the tubular body member. The method may further include engaging a portion of the suture with the locking slot to hold the suture. A majority of the surface of the tubular body member may be textured. The tubular body member may be a cylindrical body member such that the cylindrical body member does not have edges. The suture puller may be less than approximately eight inches long from an end of the first conical end member to an end of the second conical end member. The suture puller may be less than approximately 6 inches long from the end of the first conical end member to the end of the second conical end member. The suture puller may be approximately 4.5 inches long from the end of the first conical end member to the end of the second conical end member.

### BRIEF DESCRIPTION OF THE DRAWINGS

FIG. 1 illustrates a perspective view of a suture puller.

FIG. 2 is a side schematic view of the suture puller.

FIG. 3A is a cross-section schematic view of the suture puller.

FIG. 3B is a close up cross-section view of a portion of the suture puller.

FIGS. 4A and 4B are perspective views of the suture puller with sutures.

FIGS. 5A and 5B are schematic views of the suture puller in use during an ACL surgery

### DETAILED DESCRIPTION

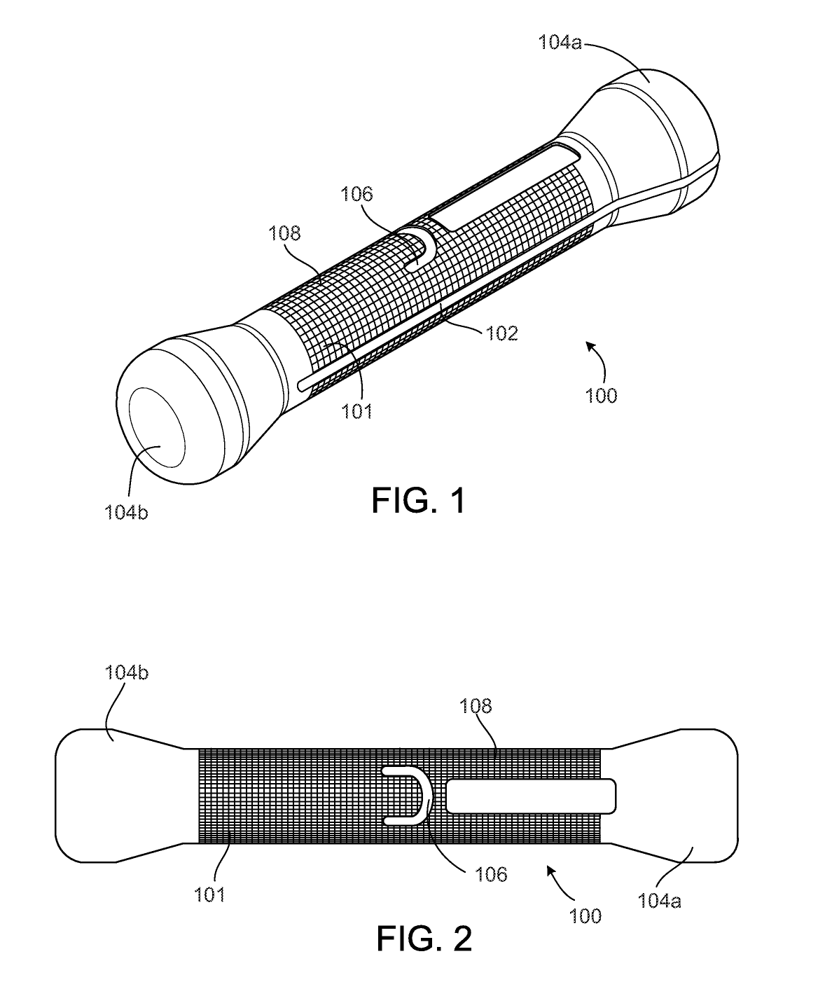

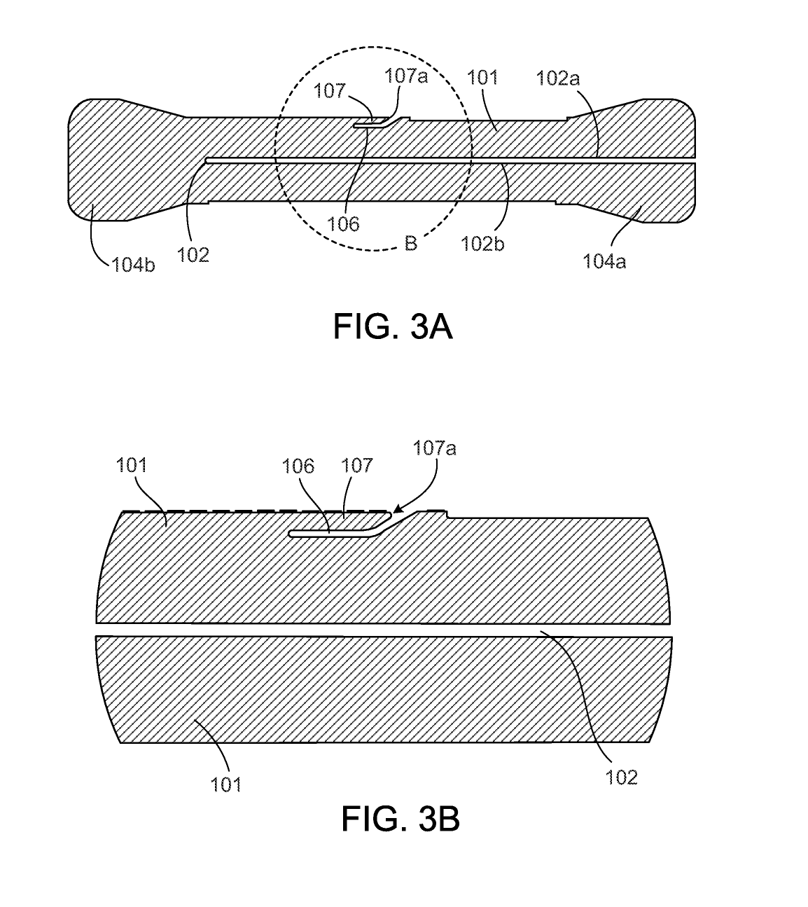

FIGS. 1-3B illustrate an example of a suture puller 100, for use in, for example, an anterior cruciate ligament (ACL) surgery. The suture puller 100 includes a tubular body member 101 with a first conical end member 104a and a second conical end member 104b. The first end member 104a is coupled to a first end of the tubular body member 101 and the second end member 104b is coupled to a second end of the tubular body member 101. During use, the larger ends may help keep the surgeon's hands from slipping off the ends of the suture puller 100.

An end member 104a and the tubular body member 101 define a slot 102 that extends through the end member 104a and at least a portion of the tubular body member 101 along a central axis of the suture puller 100. The slot 102 traverses the length of the first end member 104a and the tubular body member 101 and spans the width of the tubular body member 101 and the first end member 104a along the central axis. The slot 102 is configured to receive at least one suture (not shown). Slot 102 is configured to be at least partially compressible such that when a suture is placed within the slot 102, the suture can be compressed between the top surface 102a and the bottom surface 102b of the slot 102. This compression can hold the suture in place (that is, stop motion of the suture in the slot 102) when force is applied to the suture. In addition to the compression of the slot 102, the top surface 102a and bottom surface 102b of the slot 102 may have a lining such as rubber or silicon to limit or prevent suture slipping.

A majority of the outer surface of the tubular body member 101 includes a surface texture 108, such as cross-hatching or raised lines on the surface. As the suture is wrapped around the outer surface of the tubular body member 101, the texture 108 on the outer surf may help prevent the suture from moving. The texture 108 on the outer surface of the tubular body member 101 may also be designed to aid the surgeon in gripping the suture puller 100 without having sharp edges or features that might damage a surgeon's gloves.

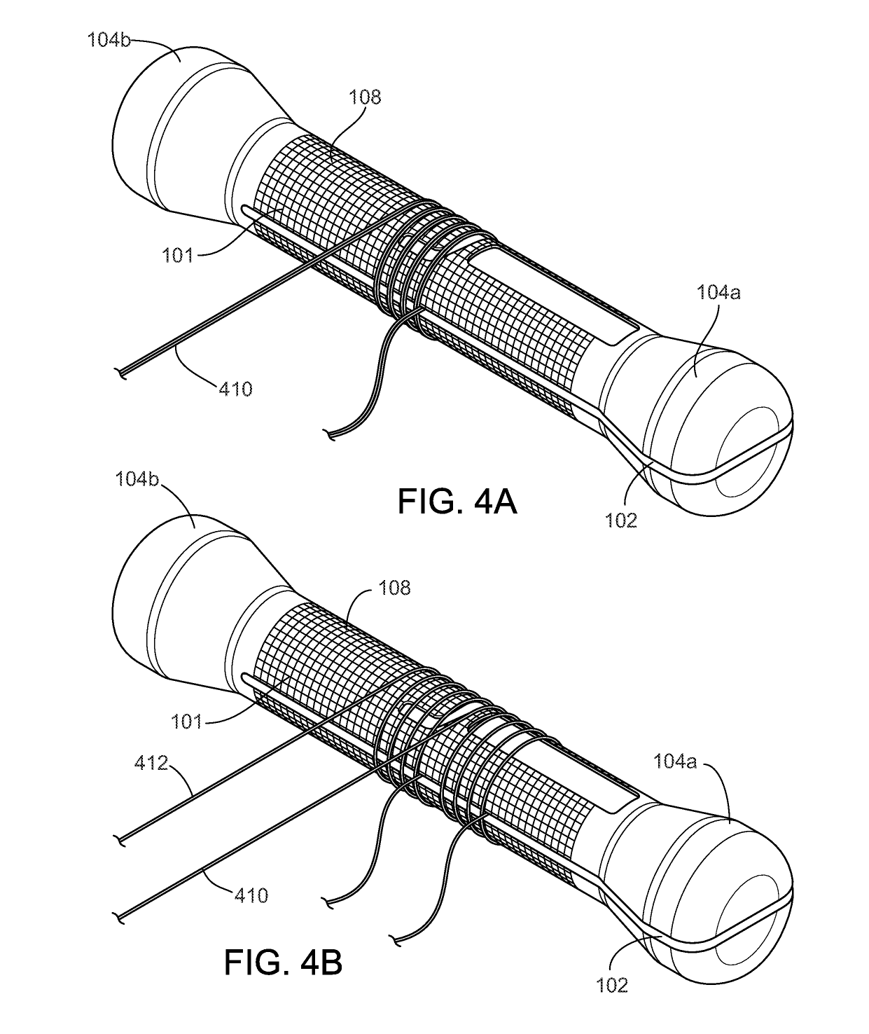

A locking slot 106 is also defined in the outer surface of the tubular body member 101, and is configured to receive and hold part of the suture. The locking slot 106 is defined by a tab 107 which extends from the outer surface of the tubular body member 101 approximately half way between the first end member 104a and the second end member 104b, such that one end 107a of the tab 107 is free (that is, not connected to the tubular body member 101. The tab 107 is rectangular with the free end 107a being rounded. The locking slot 106, as shown in FIG. 3B, on the outer surface of the tubular body member 101 may be used to hold a portion of the suture. The locking slot 106 may, for example, hold an end of a suture to additionally help keep the suture in place. The locking slot 106 may be rigid, or may be flexible to act as a spring.

The tubular body member 101 is a cylindrical body member such that the body member 101 does not have edges. This shape may minimize the risk of cutting or damaging the suture or the surgeon's gloves. More generally, the suture puller 100 may be designed such that the suture puller 100 does not have any sharp edges, which may help minimize the risk of cutting or damaging the suture or the surgeon's gloves and/or may be more comfortable for the surgeon during use.

The suture puller 100 is sized and dimensioned to fit in a surgeon's hand. The suture puller 100 may be designed to be used with one or two hands. Accordingly, in one implementation, the length of the suture puller 100 may be less than about 8 inches long from the end of one end member 104a to the end of the other end member 104b. In other implementations, the length of the suture puller 100 may be less than about 6 inches long from the end of one end member 104a to the end of the other end member 104b. In yet other implementations, the length of a suture puller 100 is about 4.5 inches long from the end of one end member 104a to the end of the other end member 104b. The symmetric design of the suture puller 100 may help a surgeon to pull the suture or graft evenly and give leverage to, for example, pull a graft attached to a suture into a bone tunnel.

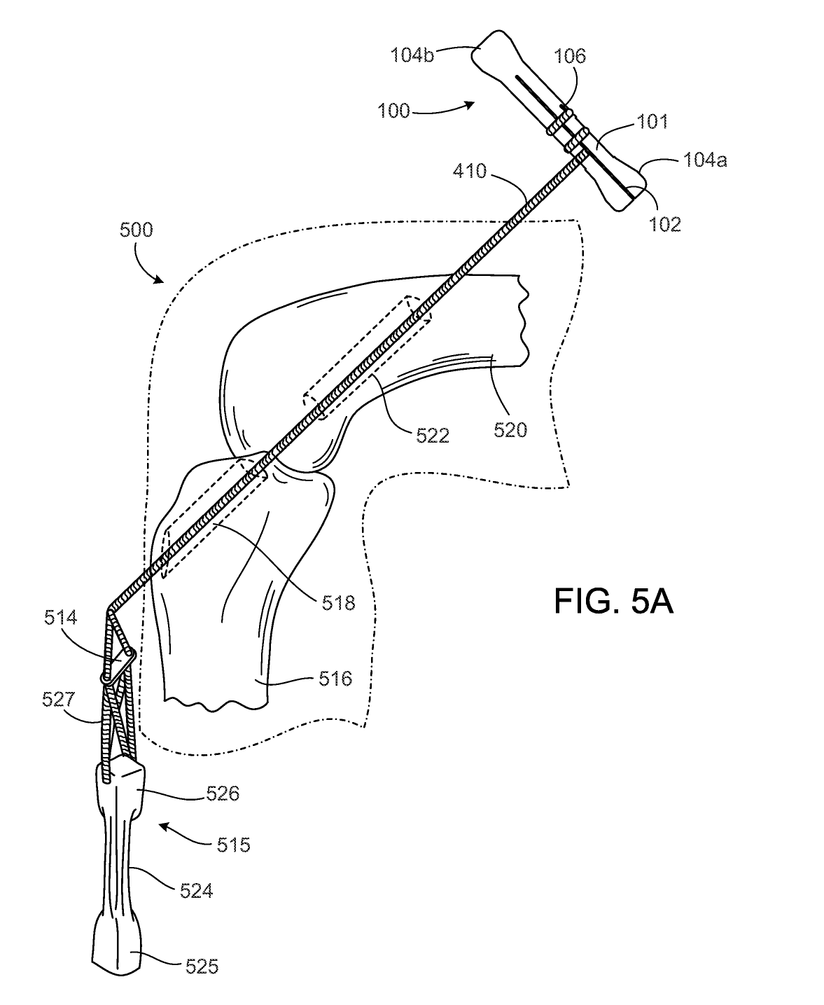

Referring to FIG. 4A a flexible member 410, such as a suture, passes through the longitudinal slot 102 defined in the tubular body member 101 and an end member 104a of the suture puller 100 and wraps around the body member 101.

During use (for example, during an ACL surgery), the surgeon passes a portion of the suture 410 through the longitudinal slot 102 in the body member 101 while the suture puller 100 rests on a surface (e.g. the surgeon's hand or fingertips). The surgeon wraps the suture around the body member 101 and then curls his fingers around the suture puller 100 to grip the suture puller. The suture 410 may pass through the surgeon's fingers, to the side of one of the surgeon's hands, or between the surgeon's hands (for example, if he is using two hands). As the surgeon grips the suture puller 100, the longitudinal slot 102 is at least partially compressed. As described above, this compression can act to secure the suture 410 within the slot 102 and limit slipping. During use, the surgeon may wrap the suture 410 around the body member 101 by, for example, rotating the suture puller 100 about its longitudinal axis. Multiple sutures 410 and 412 may be used with the suture puller 100 as shown in FIG. 4B, to facilitate procedures such as, for example, pulling an endobutton through a bone tunnel in which multiple sutures (pulling suture and trailing sutures) are attached to the endobutton.

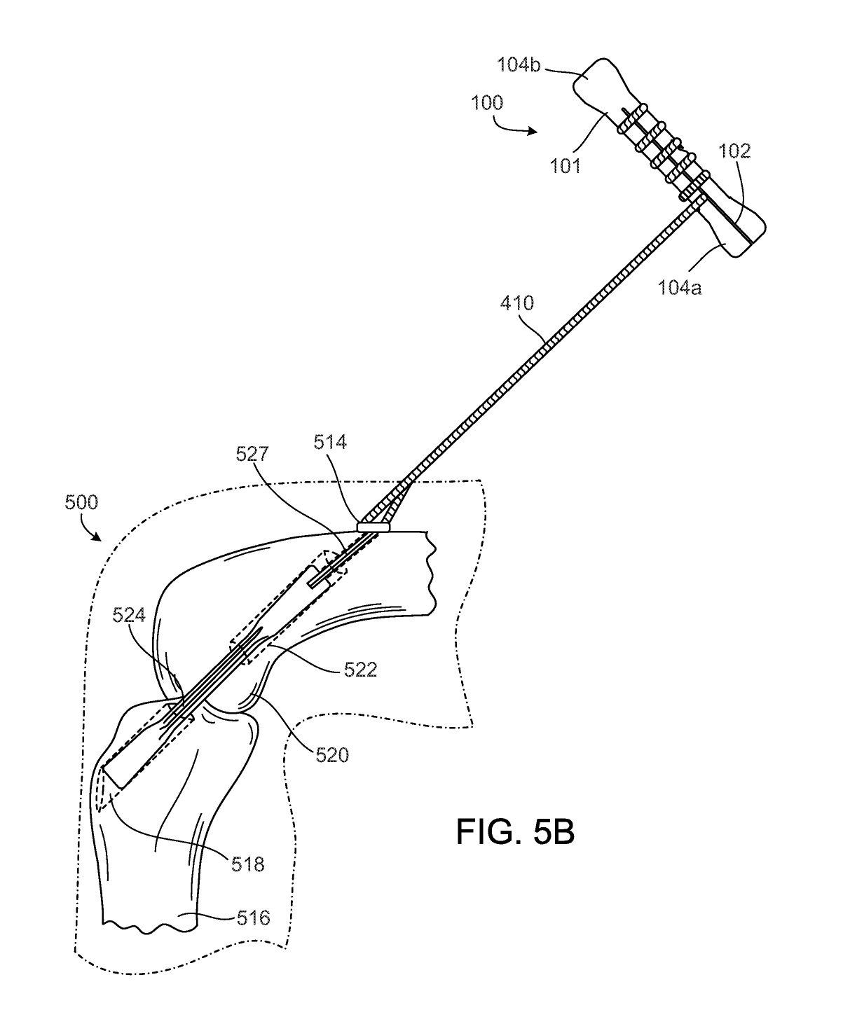

Referring to FIGS. 5A and 5B a tissue graft 524 is shown being implanted within a knee 500 during an anterior cruciate ligament (ACL) repair and reconstruction procedure. Drilling procedures are performed to form one or more bone tunnels, such as the appropriately sized tibial tunnel 518 extending through tibia 516 and femoral tunnel 522 through the femur 520. Surgical construct 515 includes a tissue graft 524, bone blocks 525 and 526, a closed-loop suture 527, and a graft attachment device 514. Tissue graft 524 has bone blocks 525, 526 on both ends, sized and shaped to fit within femoral tunnel 522 and tibial tunnel 518, respectively. The tissue graft 524 is coupled to the graft attachment device 514 by inserting a closed-loop suture 527 into bone block 526 and capturing the suture 527 within graft attachment device 514.

A first end of the suture 410 is coupled to graft attachment device 514 and a second end of the suture 410 is passed through the tunnels 518 and 522 with, for example, a passing pin (not shown). The second end of the suture 410 is withdrawn beyond the skin using a passing pin (not shown).

The second end of the suture 410 is coupled to the suture puller 100 for example, by placing the second end of the suture 410 into the longitudinal slot 102 in the suture puller 100. The surgeon grips the suture puller 100, as described above, thereby compressing slot 102 to maintain the suture 410 within the slot 102, and may also wrap a portion of the suture 410 around the tubular body member 101 after placing the second end of the suture 410 into the slot 102. A portion of the suture 410 may be engaged with the locking slot 106 to hold the suture 410. Engaging the suture 410 with the locking slot 106 may help to prevent rotation of the suture puller 100 in the surgeon's hand. The surgeon then manipulates the suture puller 100 to draw the suture, graft attachment device, and tissue graft through the bone tunnel. For instance, the surgeon may pull the suture puller 100 to draw suture 410 through tibial tunnel 518 and femoral tunnel 522, with the suture 410 pulling the graft attachment device 514 and the tissue graft 524 through the bone tunnels 518, 522 so that the tissue graft 524 is positioned within the femoral tunnel 522 and tibial tunnel 518. As the construct 515 is pulled through the tunnels 518, 522, the surgeon may wrap the suture 410 around the suture puller 100 to take up slack in the suture 410 (FIG. 5B) by, for example, rotating the suture puller 100 about its longitudinal axis. The surgeon can keep closed-loop suture 527 captured within the graft attachment device 514 by taking up any slack in suture 410 while pulling on surgical construct 515. However, the surgeon should carefully apply even tension on suture(s) 410 to position the surgical construct 515 properly. The symmetric design of the suture puller 100 can allow for even force distribution on the surgical construct 515.

Once graft attachment device 514 has been pulled through femoral tunnel 522, the surgeon positions graft attachment device 514 transversely to the femoral tunnel 522 and across the opening. Graft attachment device 514 is secured against the femur 520 by attaching tissue graft 524 to tibia 516 and tensioning tissue graft 524 and closed-loop suture 527.

A number of implementations have been described. Nevertheless, it will be understood that various modifications may be made. For example, while the suture puller 100 is shown in use with a knee joint, the suture puller can be used in other areas of the body, for example a hip or shoulder joint, and would be sized appropriately. In another example, the elongated tubular body member 101 and enlarged end pieces 104a and 104b may be shaped differently than describe, for example in an oval or spheroid shapes. The suture puller may be made out of many different materials such as stainless steel, aluminum, PEEK, a polycarbonate, acetal, etc, and may be intended for one time use or multiple uses. Although the suture puller 100 has been described for use by hand, it is contemplated that the suture puller 100 may be used with another instrument or may be connected to a pulling or winding device. Accordingly, other implementations are within the scope of the following claims.

1. A suture puller comprising:
a tubular body member having a first end and a second end;
a first conical end member coupled to the first end of the tubular body member; and
a second conical end member coupled to the second end of the tubular body member;
wherein the first conical end member and the tubular body member define a slot that extends through the first conical end member and at least a portion of the tubular body member along a central axis of the suture puller, the slot being configured to receive a suture.

2. The suture puller of claim 1 wherein the tubular body member defines a locking slot on a surface of the tubular body member, the locking slot being configured to receive and hold part of the suture.

3. The suture puller of claim 1 wherein a majority of the surface of the tubular body member is textured.

4. The suture puller of claim 1 wherein the slot is lined with a material configured to prevent slipping of the suture.

5. The suture puller of claim 1 wherein the slot is configured to be compressed such that the compression stops motion of the suture in the slot.

6. The suture puller of claim 1 wherein the tubular body member is a cylindrical body member such that the cylindrical body member does not have edges.

7. The suture puller of claim 1 wherein the suture puller is less than approximately eight inches long from an end of the first conical end member to an end of the second conical end member.

8. The suture puller of claim 7 wherein the suture puller is less than approximately 6 inches long from the end of the first conical end member to the end of the second conical end member.

9. The suture puller of claim 8 wherein the suture puller is approximately 4.5 inches long from the end of the first conical end member to the end of the second conical end member.

10. A method comprising:
forming a bone tunnel;
coupling a tissue graft to a graft attachment device
coupling a first end of a suture to the graft attachment device;
passing a second end of the suture through the bone tunnel;
coupling the second end of the suture to a suture puller, the suture puller including a tubular body member having a first end and a second end, a first conical end member coupled to the first end of the tubular body member, and a second conical end member coupled to the second end of the tubular body member; and
manipulating the suture puller to draw the suture, graft attachment device, and tissue graft through the bone tunnel

11. The method of claim 10 wherein:
the first conical end member and the tubular body member define a slot that extends through the first conical end member and at least a portion of the tubular body member along a central axis of the suture puller; and
coupling the second end of the suture to the suture puller comprises placing the second end of the suture into the slot.

12. The method of claim 11 wherein coupling the second end of the suture to the suture puller comprises wrapping a portion of the suture around the tubular body member after placing the second end of the suture in the slot.

13. The method of claim 11 comprising compressing the slot such that the compression stops motion of the suture in the slot.

14. The method of claim 11 wherein the slot is lined with a material configured to prevent slipping of the suture.

15. The method of claim 10 wherein the suture puller comprises a locking slot on a surface of the tubular body member, the method further comprising engaging a portion of the suture with the locking slot to hold the suture.

16. The method of claim 10 wherein a majority of the surface of the tubular body member is textured.

17. The method of claim 10 wherein the tubular body member is a cylindrical body member such that the cylindrical body member does not have edges.

18. The method of claim 10 wherein the suture puller is less than approximately eight inches long from an end of the first conical end member to an end of the second conical end member.

19. The method of claim 18 wherein the suture puller is less than approximately 6 inches long from the end of the first conical end member to the end of the second conical end member.

20. The method of claim 19 wherein the suture puller is approximately 4.5 inches long from the end of the first conical end member to the end of the second conical end member.
